(19) United States
(12) Patent Application Publication
(10) Pub. No.: US 2026/0187824 A1
(43) Pub. Date: Jul. 2, 2026

(54) IMAGE REGISTRATION METHOD, DOSAGE DETERMINATION METHOD, AND COMPUTER-READABLE STORAGE MEDIUM

(71) Applicant: Shanghai United Imaging Healthcare Co., Ltd., Shanghai (CN)

(72) Inventors: Lin Zhang, Shanghai (CN); Hongcheng Yang, Shanghai (CN); Yida He, Shanghai (CN); Zhi Shi, Shanghai (CN); Yanfang Liu, Shanghai (CN)

(21) Appl. No.: 19/437,541

(22) Filed: Dec. 31, 2025

(30) Foreign Application Priority Data
Dec. 31, 2024 (CN) 202411999585.2

(51) Int. Cl. G06T 7/33; A61N 5/10; G06T 3/40; G06T 7/00; G06V 10/25; G06V 10/74

(52) U.S. Cl. CPC G06T 7/337; A61N 5/1038; A61N 5/1039; G06T 3/40; G06T 7/0012; G06V 10/25; G06V 10/761; A61N 5/1045; G06T 2207/30004; G06V 2201/03

(57) ABSTRACT

The present disclosure relates to an image registration method, a dosage determination method, and a non-transitory computer-readable storage medium. The method comprises: determining first target registration information based on a first region of interest in a first floating image and a second region of interest in a first reference image; and determining second target registration information based on the first target registration information, the first floating image, and the first reference image. The second region of interest corresponds to the first region of interest. The first target registration information is used for registering the first region of interest with the second region of interest. The second target registration information is used for registering the first floating image with the first reference image.

### CROSS-REFERENCE TO RELATED APPLICATIONS

This application claims priority to the Chinese Patent Application No. 202411999585.2, filed on Dec. 31, 2024, entitled “IMAGE REGISTRATION METHOD AND COMPUTER-READABLE STORAGE MEDIUM”, and the content of which is hereby incorporated by reference in its entirety.

### TECHNICAL FIELD

The present disclosure relates to the field of medical technology, and in particular, to an image registration method, a dosage determination method, and a computer-readable storage medium.

### BACKGROUND

Image registration can be applied to target volume transfer, dose accumulation, or other aspects of adaptive radiotherapy. Therefore, image registration is an important technical means in the medical field.

### SUMMARY

In a first aspect, the present disclosure provides an image registration method including:
determining first target registration information based on a first region of interest in a first floating image and a second region of interest in a first reference image, where the second region of interest corresponds to the first region of interest, and the first target registration information is configured for registering the first region of interest with the second region of interest; and
determining second target registration information based on the first target registration information, the first floating image, and the first reference image, where the second target registration information is configured for registering the first reference image with the first floating image.

In a second aspect, the present disclosure further provides an image registration apparatus, including:
a first determination module, configured to determine first target registration information based on a first region of interest in a first floating image and a second region of interest in a first reference image, where the second region of interest corresponds to the first region of interest, and the first target registration information is configured for registering the first region of interest and the second region of interest;
a second determination module, configured to determine second target registration information based on the first target registration information, the first floating image, and the first reference image, where the second target registration information is configured for registering the first floating image and the first reference image.

In a third aspect, the present disclosure further provides a dosage determination method, including:
obtaining a first subfield corresponding to a new plan;
determining a subfield evaluation result of the first subfield based on the first subfield corresponding to the new plan and a second subfield corresponding to an old plan, where the first subfield corresponds to the second subfield;
determining dose information of the new plan based on the subfield evaluation result of the first subfield.

In a fourth aspect, the present disclosure further provides a dosage determination apparatus, including:
an obtainment module, configured to obtain a first subfield of a new plan;
a first determination module, configured to determine a subfield evaluation result of the first subfield based on the first subfield of the new plan and a second subfield of an old plan, where the first subfield corresponds to the second subfield;
a second determination module, configured to determine dose information of the new plan based on the subfield evaluation result of the first subfield.

In a fifth aspect, the present disclosure further provides a computer device, including a memory and a processor, the memory storing a computer program. The processor, when executing the computer program, implements the above methods.

In a sixth aspect, the present disclosure further provides a non-transitory computer-readable storage medium having a computer program stored thereon, and the computer program, when executed by a processor, implements the above methods.

In a seventh aspect, the present disclosure further provides a computer program product, including a computer program. The computer program, when executed by a processor, implements the above methods.

### BRIEF DESCRIPTION OF THE DRAWINGS

FIG. 1 is a diagram of an application environment of the present disclosure.

FIG. 2 is a schematic flowchart of an image registration method in an embodiment.

FIG. 3 is a schematic flowchart of a method for determining first target registration information in an embodiment.

FIG. 4 is a schematic flowchart of another method for determining first target registration information in an embodiment.

FIG. 5 is a schematic flowchart of yet another method for determining first target registration information in an embodiment.

FIG. 6 is a schematic flowchart of still another method for determining first target registration information in an embodiment.

FIG. 7 is a schematic flowchart of another method for determining first target registration information in an embodiment.

FIG. 8 is a schematic flowchart of yet another method for determining first target registration information in an embodiment.

FIG. 9 is a schematic flowchart of a method for determining second target registration information in an embodiment.

FIG. 10 is a schematic flowchart of another method for determining second target registration information in an embodiment.

FIG. 11 is a schematic flowchart of yet another method for determining second target registration information in an embodiment.

FIG. 12 is a schematic flowchart of a dosage determination method in an embodiment.

FIG. 13 is a schematic flowchart of a method for determining dose information of a new plan in an embodiment.

FIG. 14 is a schematic flowchart of a dosage determination method in an embodiment.

FIG. 15 is a structural block diagram of an image registration apparatus in an embodiment.

FIG. 16 is a structural block diagram of a dosage determination apparatus in an embodiment.

### DETAILED DESCRIPTION

To make the objectives, technical solutions, and advantages of the present disclosure clearer, the present disclosure will be further described in detail below with reference to the accompanying drawings and embodiments. The embodiments described herein are only used to explain the technical solutions disclosed in the present disclosure, but are not to limit the technical solutions.

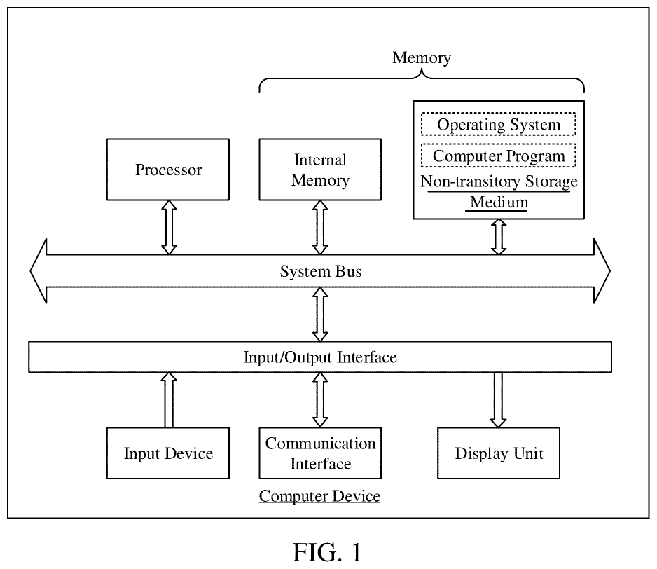

In an embodiment, as shown in FIG. 1, a computer device is provided. The computer device may be a terminal or a server, and an internal structure thereof can be as shown in FIG. 1. The computer device includes a processor, a memory, an input/output (I/O) interface, and a communication interface. The processor, the memory, and the input/output interface are connected through a system bus. The communication interface is connected to the system bus through the input/output interface. The processor of the computer device is configured to provide computing and control capabilities. The memory of the computer device includes a non-transitory storage medium and an internal memory. The non-transitory storage medium stores an operating system, a computer program, and a database. The internal memory provides an operating environment for the operating system and the computer program in the non-transitory storage medium. The database of the computer device is configured to store relevant data. The input/output interface of the computer device is configured to exchange information between the processor and an external device. The communication interface of the computer device is configured to communicate with an external terminal through a network connection. The computer program implements an image registration method when executed by the processor.

The structure shown in FIG. 1 is merely a structure involved in a technical solution of the present disclosure. This structure does not constitute the only definition of the computer device used in the technical solutions of the present disclosure. Compared with the structure shown in FIG. 1, the computer device in the technical solutions of the present disclosure may further include more components, omit some components, or have different component arrangements.

In an embodiment, the image registration method is applied to a server or a terminal, and can also be applied to a system including a terminal and a server. The image registration method is implemented through interaction between the terminal and the server. The terminal may include, but is not limited to, a personal computer, a laptop computer, a smartphone, or a tablet computer. The server may include an independent server or a server cluster composed of multiple servers.

Since image registration can be applied to target volume transfer, dose accumulation, or other aspects of adaptive radiotherapy, image registration is an important technical means in the medical field. However, image registration in the related art is a highly underdetermined optimization. Therefore, in the related art, the accuracy of registration is poor, especially when there are large differences between various regions of different images, leading to difficulty in fully registering these images. Therefore, improving the accuracy of image registration is a critical technical problem to be addressed in the field. Accordingly, it is necessary to provide an image registration method with relatively high accuracy to address the above technical problem.

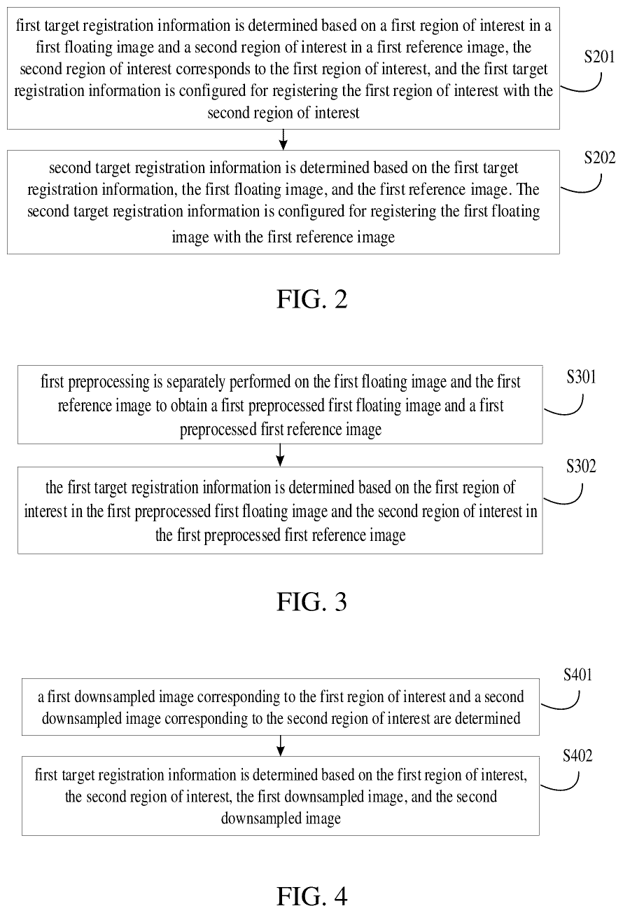

In an embodiment, as shown in FIG. 2, an image registration method is provided. Taking the method applied to the computer device in FIG. 1 as an example, the image registration method includes the following Step S201 and Step S202.

In Step S201, first target registration information is determined based on a first region of interest in a first floating image and a second region of interest in a first reference image. The second region of interest corresponds to the first region of interest. The first target registration information is used for registering the first region of interest with the second region of interest.

In this embodiment, the computer device can obtain the first floating image and the first reference image. At least a part of the first floating image includes the first region of interest. At least a part of the first reference image includes the second region of interest. For example, the first floating image includes the first region of interest and a first surrounding region located around the first region of interest. The first reference image includes the second region of interest and a second surrounding region located around the second region of interest.

In an embodiment, the registration information can include deformable registration information for deformable registration or rigid registration information for rigid registration. The present disclosure mainly takes the registration information as deformable registration information as an example for explanation, but those skilled in the art can understand that, based on the knowledge in the field, adaptive adjustments can be made to make the rigid registration information also applicable to the technical solution involved in the present disclosure. For example, when the difference between the first floating image and the first reference image is small, the difference between the first floating image and the first reference image can be regarded as a simple translation or displacement, and the technical solution described in the present disclosure can be used to perform rigid registration on the first floating image and the second floating image.

In an embodiment, the first floating image can be an original floating image or an image obtained after preprocessing the original floating image. The first reference image can be an original reference image or an image obtained after preprocessing the original reference image.

In an embodiment, the preprocessing includes, but is not limited to, various processes such as window parameter adjustment and/or pixel value normalization. For example, the computer device can take two medical images to be registered as the first floating image and the first reference image, respectively.

In an embodiment, the first floating image and the first reference image can be images of a subject at different phases. For example, the first reference image may be a medical image at an end of inspiration, and the first floating image may be a medical image at an end of expiration.

In an embodiment, the medical image may include, but is not limited to, a Computed Tomography (CT) image, a Positron Emission Computed Tomography (PET) image, a Magnetic Resonance (MR) image, a Positron Emission Tomography-Computed Tomography (PET-CT) image, or a Positron Emission Tomography-Magnetic Resonance (PET-MR) image.

In an embodiment, the computer device can determine the first region of interest in the first floating image and the second region of interest in the first reference image. The first region of interest corresponds to the second region of interest. In other words, the computer device can determine N first regions of interest in the first floating image, and N second regions of interest in the first reference image that are in one-to-one correspondence with the N first regions of interest. N is an integer greater than or equal to 1.

In an embodiment, each of the first region of interest and the second region of interest is a region of interest (ROI) in their respective corresponding images. Taking radiotherapy as an example, the region of interest may include various physiological structures, such as tissues, parts, organs, lesions, or combinations thereof, in a corresponding image.

In an embodiment, the first region of interest can be a radiated target region of a subject (for example, a lesion of a patient) at a first time. The second region of interest can be the same radiated target region of the object at a second time. The first time differs from the second time. The first region of interest and the second region of interest may be the same or have a certain degree of difference. For example, in radiotherapy, due to disease progression or the effect of radiotherapy, the first region of interest may be larger or smaller than the second region of interest.

In an embodiment, the computer device may take a liver region in the first floating image as the first region of interest 1, and a blood vessel region in the first floating image as the first region of interest 2. Similarly, the computer device will take a liver region in the first reference image as the second region of interest 1, and a blood vessel region in the first reference image as the second region of interest 2.

In an embodiment, the computer device may use a preset segmentation algorithm or a segmentation model to segment the image for contouring the regions of interest. Alternatively, the user may determine the first region of interest and the second region of interest through manual segmentation or contouring. The embodiments of the present disclosure do not limit the above approach of segmentation or contouring.

In an embodiment, the computer device may determine the first target registration information based on the first region of interest and the second region of interest. The first target registration information is used for registering the region of interest in the first floating image with the region of interest in the first reference image. The first target registration information includes, but is not limited to, a displacement field, a deformation field, a mean value or standard deviation of pixel values, or other registration information that can be used for registering the first region of interest and the second region of interest. The first target registration information can at least register the first region of interest and the second region of interest.

In some embodiments, the first target registration information can also register a region other than the first region of interest in the first floating image and a region other than the second region of interest in the first reference image.

In an embodiment, the computer device may use a first preset deformation algorithm to register the first region of interest and the second region of interest to obtain the first target registration information. The first preset deformation algorithm includes, but is not limited to, a Free Form Deform (FFD) algorithm or a Demons algorithm, and the embodiments of the present disclosure do not limit the selection of the preset deformation algorithm.

The following describes an example where the computer device obtains the first target registration information by registering the first region of interest and the second region of interest according to an optimization objective F.

In an embodiment, the optimization objective F can be determined according to Formula (1) as follows:

$$F = arg {min}_{F} ( \begin{matrix}α {‖ {I}_{ref} - D ( {I}_{mov} ) ‖}_{2}^{2} + {\sum }_{i = 1}^{k} {β}_{i} {‖ {ROI}_{ref}^{i} - D ( {ROI}_{mov}^{i} ) ‖}_{2}^{2} + \\ γ {‖ D - {D}_{ref} ‖}_{2}^{2}\end{matrix} ) \qquad ( 1 )$$

where, α represents a first weight, and k represents the number of regions of interest participating in a calculation. For example, k may be a number less than or equal to N. βᵢ represents a second weight corresponding to the i-th region of interest among the k regions of interest. γ represents a third weight. α, βᵢ, and γ can be set according to requirements, α and βᵢ are numbers greater than or equal to 0. γ is a number greater than 0.

$${‖ ‖}_{2}^{2}$$

represents a square of an L2 norm. k represents determined according to actual requirements.

I_{ref} represents a reference image. Iₘₒᵥ represents a floating image.

$${ROI}_{ref}^{i}$$

represents the i-th region of interest in the reference image.

$${ROI}_{mov}^{i}$$

represents the i-th region of interest in the floating image. The value range i is of [1, k].

D_{ref} represents reference registration information. D represents current registration information, that is, the registration information to be solved. In other words, the computer device can find a better current registration information D according to the optimization objective F.

In an embodiment, let α=0. The computer device substitutes the first region of interest into

$${ROI}_{mov}^{i} ,$$

substitutes the second region of interest into

$${ROI}_{ref}^{i} ,$$

and assigns an initial value (for example, 0) to D_{ref}. Then, the computer device iterates the current registration information D based on Formula (1), and takes D as the first target registration information when a stop condition is met. The stop condition can be that the number of iterations reaches a preset number, or the optimization objective F is less than a preset threshold. The embodiments of the present disclosure do not specifically limit the stop condition. The stop condition can be determined according to a specific requirement.

In an embodiment, the computer device may also determine the first target registration information based on the first region of interest, the second region of interest, the first floating image, and the first reference image. Referring to Formula (1), let α≠0. The computer device may substitute the first reference image into I_{ref}, substitute the first floating image into Iₘₒᵥ, substitute the first region of interest into

$${ROI}_{mov}^{i} ,$$

substitute the second region of interest into

$${ROI}_{ref}^{i} ,$$

and assign an initial value (for example, 0) to D_{ref}. Then, the computer device iterates the current registration information D based on Formula (1), and takes the current registration information D as the first target registration information when the stop condition is met.

In Step S202, second target registration information is determined based on the first target registration information, the first floating image, and the first reference image. The second target registration information is used for registering the first floating image with the first reference image.

After obtaining the first target registration information, the computer device can determine the second target registration information based on the first target registration information, the first floating image, and the first reference image. The second target registration information is used for registering the first floating image with the first reference image.

In an embodiment, the first target registration information includes deformable registration information for registering the region of interest of the first floating image with the region of interest of the first reference image. The second target registration information includes deformable registration information for registering the entire first floating image with the entire first reference image, or for registering a first part and a second part. The first part is a part of the first floating image that contains the first region of interest, but has a larger size than the first region of interest. The second part is a part of the first reference image corresponding to the first part. For example, the computer device can perform deformation on the first reference image according to the second target registration information to obtain a processed first reference image, so as to achieve the purpose of registering the first floating image with the first reference image. The second target registration information includes, but is not limited to, a displacement field, a deformation field, a mean value or standard deviation of pixel values, or other registration information that can be used for registering the first floating image with the first reference image.

In an embodiment, the computer device can use a second preset deformation algorithm and the first target registration information to register the first floating image and the first reference image to obtain the second target registration information. The second preset deformation algorithm may also include, but is not limited to, an FFD algorithm or a Demons algorithm. The second preset deformation algorithm may be the same as or different from the first preset deformation algorithm.

In an embodiment, the first preset deformation algorithm may be an FFD algorithm with a large degree of deformation. The second preset deformation algorithm may be a Demons algorithm with a small degree of deformation.

The following describes an example where the computer device obtains the second target registration information according to the optimization objective F by registering the first floating image and the first reference image on the basis of the first target registration information.

In an embodiment, referring to Formula (1), let βᵢ=0. The computer device substitutes the first reference image into I_{ref}, substitutes the first floating image into Iₘₒᵥ, substitutes the first target registration information into D_{ref}, iterates the current registration information D based on Formula (1), and takes the current registration information D as the second target registration information when the stop condition is met.

In the above image registration method, the first target registration information is determined based on the first region of interest in the first floating image and the second region of interest in the first reference image. The second region of interest corresponds to the first region of interest. Since the first target registration information is used for registering the first region of interest with the second region of interest, the first target registration information obtained through the registration of the regions of interest is focused on in the first place. Then, the second target registration information is determined based on the first target registration information, the first floating image, and the first reference image. As such, on the basis of the first target registration information, registration can be performed based on the entire first floating image and the entire first reference image, so that not only the regions of interest of the two images can be well registered, but also the parts other than the regions of interest in the two images can be well registered. Thus, the first floating image and the first reference image can be accurately registered through the second target registration information.

In an embodiment, as shown in FIG. 3, Step S201 includes Step S301 and Step S302.

In Step S301, first preprocessing is separately performed on the first floating image and the first reference image to obtain a first preprocessed first floating image and a first preprocessed first reference image.

In this embodiment, the first preprocessing includes, but is not limited to, processing such as window parameter adjustment and/or pixel value normalization. A window parameter may include a window level or a window width. In this way, after performing the first preprocessing on the first floating image, the first preprocessed first floating image can be obtained. After performing the first preprocessing on the first reference image, the first preprocessed first reference image can be obtained.

In Step S302, the first target registration information is determined based on the first region of interest in the first preprocessed first floating image and the second region of interest in the first preprocessed first reference image.

In an embodiment, the computer device may take the liver region in the first preprocessed first floating image as the first region of interest 1, and the blood vessel region in the first preprocessed first floating image as the first region of interest 2. Similarly, the computer device may take the liver region in the first preprocessed first reference image as the second region of interest 1, and the blood vessel region in the first preprocessed first reference image as the second region of interest 2.

In an embodiment, the computer device may determine the first target registration information based on the first region of interest in the first preprocessed first floating image and the second region of interest in the first preprocessed first reference image.

Determining the first target registration information in Step S302 is similar to that in Step S201, and will not be repeated here. In other words, the computer device can take the first preprocessed first floating image as a new first floating image and the first preprocessed first reference image as a new first reference image, and return to Step S201 for the new first floating image and the new first reference image to obtain the second target registration information.

In the above embodiments, since the first preprocessing can be separately performed on the first floating image and the first reference image to obtain the first preprocessed first floating image and the first preprocessed first reference image, the first floating image and the first reference image can be correspondingly preprocessed according to requirements. Thus, the first target registration information can be more efficiently determined based on the first region of interest in the first preprocessed first floating image and the second region of interest in the first preprocessed first reference image.

In an embodiment, as shown in FIG. 4, Step S201 includes Step S401 and Step S402.

In Step S401, a first downsampled image corresponding to the first region of interest and a second downsampled image corresponding to the second region of interest are determined.

In an embodiment, the computer device may separately perform downsampling on the first region of interest and the second region of interest to obtain the first downsampled image corresponding to the first region of interest and the second downsampled image corresponding to the second region of interest.

In Step S402, first target registration information is determined based on the first region of interest, the second region of interest, the first downsampled image, and the second downsampled image.

In an embodiment, the computer device can use a first preset deformation algorithm to register the first downsampled image and the second downsampled image to obtain initial registration information, and use the first preset deformation algorithm to register the first region of interest and the second region of interest on the basis of the initial registration information to obtain the first target registration information.

In the above embodiments, since the first downsampled image corresponding to the first region of interest and the second downsampled image corresponding to the second region of interest are determined, and the first target registration information is determined based on the first region of interest, the second region of interest, the first downsampled image, and the second downsampled image, the first target registration information can be determined more accurately by downsampling.

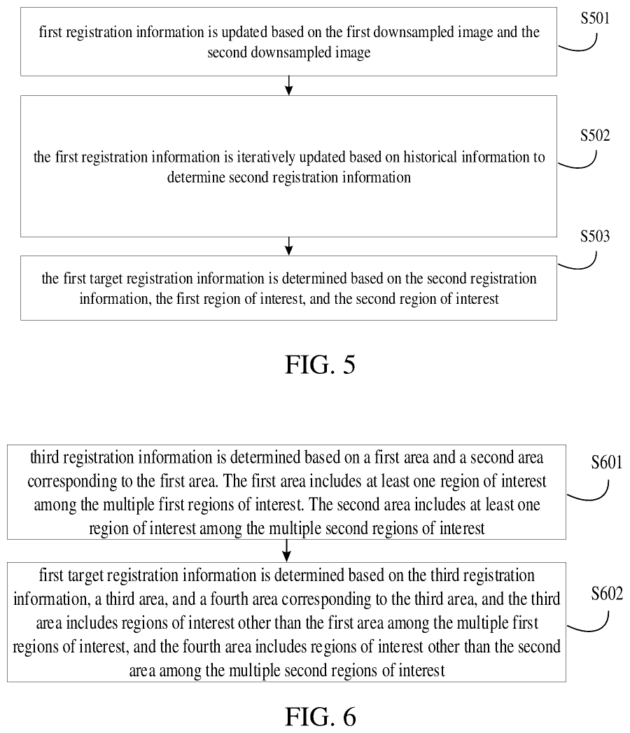

In an embodiment, as shown in FIG. 5, Step S402 includes Step S501 to Step S503.

In Step S501, first registration information is updated based on the first downsampled image and the second downsampled image.

In an embodiment, the first downsampled image and the second downsampled image may include downsampled images of different resolutions. Taking two downsampling operations as an example, the resolution of the first region of interest 1 is 400 px×400 px×100 px. After the first downsampling on the first region of interest 1, a first downsampled image A with the resolution of 200 px×200 px×50 px is obtained. The resolution of 200 px×200 px×50 px is a first resolution. After the second downsampling on the first downsampled image 1-A, a first downsampled image 1-B with the resolution of 100 px×100 px×25 px is obtained. The resolution of 100 px×100 px×25 px is a second resolution. Similarly, the computer device can also determine a second downsampled image 1-A and a second downsampled image 1-B corresponding to the second region of interest 1. px represents pixels. The processing of other first regions of interest and second regions of interest is the same as the above, and will not be repeated here.

The current resolution refers to the resolution corresponding to the first downsampled image and the second downsampled image that are currently being registered. The resolutions corresponding to different downsampled images may be different.

In an embodiment, the initial current resolution may be selected as the last resolution. The last resolution may be the smallest.

Taking the current resolution as the last resolution as an example, the computer device determines new first registration information 2 based on the first registration information 1, the first downsampled image 1-B, and the second downsampled image 1-B. The first registration information 1 can be an initial value, such as 0.

In an embodiment, referring to Formula (1), let α=0. The computer device substitutes the first downsampled image 1-B into

$$R O {I}_{mov}^{i} ,$$

substitutes the second downsampled image 1-B into

$${ROI}_{ref}^{i} ,$$

substitutes the first registration information 1 into D_{ref}, iterates the current registration information D based on Formula (1), and takes the current registration information D as the new first registration information 2 when the stop condition is met.

The above description takes one first region of interest and one second region of interest as examples. The processing of multiple first regions of interest and multiple second regions of interest is the same and will not be repeated here.

In Step S502, the first registration information is iteratively updated based on historical information to determine second registration information.

In an embodiment, for a historical resolution of the current resolution, the step of determining new first registration information based on the first registration information, the first downsampled image of the current resolution, and the second downsampled image of the current resolution is returned by using the new first registration information, until second registration information is determined based on the new first registration information, the first downsampled image of the first resolution, and the second downsampled image of the first resolution.

In an embodiment, after updating the first registration information of the current resolution, the computer device will return the historical resolution of the current resolution, and continue to update on the basis of the first registration information of the current resolution to obtain the first registration information of the last resolution through the update. The historical resolution of the current resolution can be the last resolution of the current resolution.

In an embodiment, the computer device returns the historical resolution of the current resolution. Since the historical resolution of the current resolution is the first resolution, the computer device determines the second registration information based on the first registration information 2, the first downsampled image 1-A, and the second downsampled image 1-B.

In an embodiment, referring to Formula (1), let α=0. The computer device substitutes the first downsampled image 1-A into

$${ROI}_{mov}^{i} ,$$

substitutes the second downsampled image 1-A into

$${ROI}_{ref}^{i} ,$$

substitutes the first registration information 2 into D_{ref}, iterates the current registration information D based on Formula (1), and takes the current registration information D as the second registration information when the stop condition is met.

In Step S503, the first target registration information is determined based on the second registration information, the first region of interest, and the second region of interest.

In an embodiment, the computer device can determine the first target registration information based on the second registration information, the first region of interest 1, and the second region of interest 1.

In an embodiment, referring to Formula (1), let α=0. The computer device substitutes the first region of interest 1 into

$$R O {I}_{mov}^{i} ,$$

substitutes the second region of interest 1 into

$${ROI}_{ref}^{i} ,$$

substitutes the second registration information into D_{ref}, iterates the current registration information D based on Formula (1), and takes the current registration information D as the first target registration information when the stop condition is met.

Step S501 to Step S503 are described by taking two downsampling operations as an example. The processing of other numbers of downsampling operations is the same, and will not be repeated here.

In the above embodiments, new first registration information is determined based on the first registration information, the first downsampled image of the current resolution, and the second downsampled image of the current resolution. For the historical resolution of the current resolution, the step of determining new first registration information based on the first registration information, the first downsampled image of the current resolution, and the second downsampled image of the current resolution is returned by using the new first registration information, until the second registration information is determined based on the new first registration information, the first downsampled image of the first resolution, and the second downsampled image of the first resolution. Then, the first target registration information is determined based on the second registration information, the first region of interest, and the second region of interest. As such, in the process of determining the first target registration information, iteration can be performed based on downsampled images of different resolutions, and the registration information of the next resolution serves as the basis for determining the registration information of the last resolution. Thus, relatively accurate first target registration information can be determined through continuous iteration.

In some application scenarios, when the number of first regions of interest and the number of the corresponding second regions of interest are large, or the structural differences between the regions of interest are large, likely, the first target registration information with better accuracy cannot be determined through a single processing. Therefore, the process of determining the first target registration information can be divided into several steps. In each step, the registration information for registering the first region of interest and the second region of interest is determined, and then on the basis of this registration information, the registration information for registering the first region of interest and the second region of interest is further determined, so that the first target registration information with better accuracy can be determined through multiple processings.

The following describes the process of determining the first target registration information through multiple processes. Both the first region of interest and the second region of interest can be plural. In an embodiment, as shown in FIG. 6, Step S201 includes Steps S601 to S602.

In Step S601, third registration information is determined based on a first area and a second area corresponding to the first area. The first area includes at least one region of interest among the multiple first regions of interest. The second area includes at least one region of interest among the multiple second regions of interest.

In this embodiment, the process of determining the third registration information based on the first area and the corresponding second area is similar to the processing in Step S201, and will not be repeated here.

In an embodiment, the first area includes at least one region of interest among the multiple first regions of interest. The second area includes at least one region of interest among the multiple second regions of interest. In this embodiment, there are three first regions of interest (first regions of interest I/II/III) and three second regions of interest (second regions of interest I/II/III) corresponding to the three first regions of interest, respectively. The computer device may take the first region of interest 1 as the first area and the second region of interest 1 corresponding to the first region of interest 1 as the second area to determine the third registration information based on the first area and the second area corresponding to the first area.

In an embodiment, referring to Formula (1), let α=0 and k=1. The computer device substitutes the first region of interest 1 into

$${ROI}_{mov}^{i} ,$$

substitutes the first region of interest 2 into

$${ROI}_{ref}^{i} ,$$

and assigns an initial value (for example, 0) to D_{ref}. Then, the computer device iterates the current registration information D based on Formula (1), and takes the current registration information D as the third registration information when the stop condition is met.

In Step S602, first target registration information is determined based on the third registration information, a third area, and a fourth area corresponding to the third area. The third area includes regions of interest other than the first area among the multiple first regions of interest. The fourth area includes regions of interest other than the second area among the multiple second regions of interest.

In an embodiment, the third area includes regions of interest other than the first area among the multiple first regions of interest. For example, the third area includes the regions of interest other than the first area among the multiple first regions of interest. The fourth area includes the regions of interest other than the second area among the multiple second regions of interest. For example, the fourth area includes the regions of interest other than the second area among the multiple second regions of interest. Specifically, the computer device may take the first region of interest 2 and the first region of interest 3 as the third area, and take the second region of interest 2 and the second region of interest 3 as the fourth area.

In an embodiment, the computer device may register the third area and the fourth area corresponding to the third area on the basis of the third registration information to obtain the first target registration information. For example, referring to Formula (1), let α=0 and k=2. The computer device substitutes the first region of interest 2 and the first region of interest 3 into ROIₘₒᵥⁱ, substitutes the second region of interest 2 and the second region of interest 3 into ROI_{ref}ⁱ, substitutes the third registration information into D_{ref}, iterates the current registration information D based on Formula (1), and takes the current registration information D as the first target registration information when the stop condition is met.

In some embodiments, the computer device can further divide the third area and the fourth area. First, fourth registration information is determined based on the third registration information, part of the regions of interest in the third area, and the corresponding part of the regions of interest in the fourth area. Then, on the basis of the fourth registration information, the remaining regions of interest in the third area and the corresponding remaining regions of interest in the fourth area are used to determine the first target registration information.

In the above embodiments, the first area includes at least one region of interest among the multiple first regions of interest. The second area includes at least one region of interest among the multiple second regions of interest. The third area includes the regions of interest other than the first area among the multiple first regions of interest. The fourth area includes the regions of interest other than the second area among the multiple second regions of interest. Since the third registration information can be determined based on the first area and the second area corresponding to the first region, and the first target registration information can be determined based on the third registration information, the third area, and the fourth area corresponding to the third area, the process of determining the first target registration information can be divided into several steps. The registration information determined through part of the regions of interest at each time can serve as the basis for determining the registration information through another part of the regions of interest. As such, the registration information for registering part of the regions of interest can be determined first, and then the registration information for registering the other part can be determined on this basis. By repeating this process, the relatively accurate first target registration information can be determined.

In an embodiment, Step S201 may be implemented as the first target registration information is determined based on the first region of interest, the second region of interest, the first floating image, and the first reference image.

In this embodiment, the computer device can register the first region of interest and the second region of interest, and register the first floating image and the first reference image based on the optimization objective F to obtain the first target registration information. For example, referring to Formula (1), the computer device can substitute the first reference image into I_{ref}, substitute the first floating image into Iₘₒᵥ, substitute the first region of interest into

$${ROI}_{mov}^{i} ,$$

Substitute the second region of interest into

$${ROI}_{ref}^{i} ,$$

and assign an initial value (for example, 0) to D_{ref}. Then, the computer device iterates the current deformation registration information D based on Formula (1), and takes the current deformation registration information D as the first target registration information when the stop condition is met.

In some embodiments, the computer device can also determine a sampled image corresponding to the first floating image, a sampled image corresponding to the first reference image, a first downsampled image corresponding to the first region of interest, and a second downsampled image corresponding to the second region of interest, and determine the first target registration information based on the first floating image and the sampled image corresponding to the first floating image, the first reference image and the sampled image corresponding to the first reference image, the first region of interest and the first downsampled image corresponding to the first region of interest, and the second region of interest and the second downsampled image corresponding to the second region of interest. A downsampled image is a low-resolution image corresponding to a sampled image, obtained by performing downsampling on an original image (i.e., the sampled image). The purpose of performing the downsampling on a sampled image is to reduce the complexity of data processing by reducing the number of pixels or decreasing the image size, on the premise of retaining the core features of the original image as much as possible.

In an embodiment, the computer device can use a first preset deformation algorithm to register the first downsampled image and the second downsampled image, and register the sampled image corresponding to the first floating image and the sampled image corresponding to the first reference image to obtain the initial registration information. Then, the computer device uses the first preset deformation algorithm to register the first region of interest and the second region of interest, and registers the first floating image and the first reference image on the basis of the initial registration information to obtain the first target registration information.

In an embodiment, the computer device determines new fifth registration information based on the fifth registration information, the sampled image of the current resolution, the first downsampled image of the current resolution, and the second downsampled image of the current resolution. For the historical resolution of the current resolution, the step of determining new fifth registration information based on the fifth registration information, the sampled image of the current resolution, the first downsampled image of the current resolution, and the second downsampled image of the current resolution is returned by using the new fifth registration information, until sixth registration information is determined based on the new fifth registration information, the sampled image of the first resolution, the first downsampled image of the first resolution, and the second downsampled image of the first resolution. Then, the first target registration information is determined based on the sixth registration information, the first floating image, the first reference image, the first region of interest, and the second region of interest. The first fifth registration information can be an initial value, such as 0.

In an embodiment, taking one first region of interest 1 and two downsampling operations as an example, after the first downsampling of the first floating image, the first reference image, the first region of interest 1, and the second region of interest 1, a sampled image a of the first floating image, a sampled image a of the first reference image, a first downsampled image 1-A, and a second downsampled image 1-A are obtained, respectively.

After the second downsampling of the sampled image a of the first floating image, the sampled image a of the first reference image, the first downsampled image 1-A, and the second downsampled image 1-A, a sampled image b of the first floating image, a sampled image b of the first reference image, a first downsampled image 1-B, and a second downsampled image 1-B are obtained, respectively.

In an embodiment, the current resolution is the last. Referring to Formula (1), the computer device substitutes the sampled image b of the first reference image into I_{ref}, substitutes the sampled image b of the first floating image into Iₘₒᵥ, substitutes the first downsampled image 1-B into

$${ROI}_{mov}^{i} ,$$

substitutes the second downsampled image 1-B into

$${ROI}_{ref}^{i} ,$$

substitutes the fifth registration information 1 into D_{ref}, iterates the current registration information D based on Formula (1), and takes the current registration information D as the new fifth registration information 2 when the stop condition is met.

In an embodiment, the computer device substitutes the sampled image a of the first reference image into I_{ref}, substitutes the sampled image a of the first floating image into Iₘₒᵥ, substitutes the first downsampled image 1-A into

$${ROI}_{mov}^{i} ,$$

substitutes the second downsampled image 1-A into

$${ROI}_{ref}^{i} ,$$

substitutes the fifth registration information 2 into D_{ref}, iterates the current registration information D based on Formula (1), and takes the current registration information D as the sixth registration information when the stop condition is met.

In an embodiment, the computer device substitutes the first reference image into I_{ref}, substitutes the first floating image into Iₘₒᵥ, substitutes the first region of interest 1 into

$${ROI}_{ref}^{i} ,$$

substitutes the second region of interest 1 into

$${ROI}_{mov}^{i} ,$$

substitutes the sixth registration information into D_{ref}, iterates the current registration information D based on Formula (1), and takes the current registration information D as the first target registration information when the stop condition is met.

In the above embodiments, since the first target registration information can be determined based on the first region of interest, the second region of interest, the first floating image, and the first reference image, full-image information can also be combined in the process of determining the first target registration information, thereby determining the relatively accurate first target registration information.

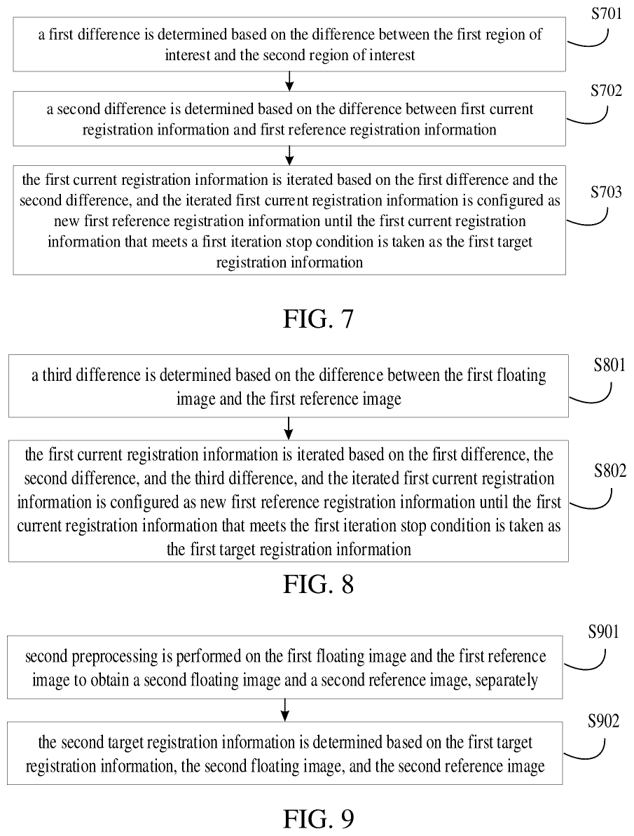

In an embodiment, as shown in FIG. 7, Step S201 includes Step S701 to Step S703.

In Step S701, a first difference is determined based on the difference between the first region of interest and the second region of interest.

In Step S702, a second difference is determined based on the difference between first current registration information and first reference registration information.

In Step S703, the first current registration information is iterated based on the first difference and the second difference, and the iterated first current registration information is used as new first reference registration information until the first current registration information that meets a first iteration stop condition is taken as the first target registration information.

In this embodiment, the above differences can include, but are not limited to, root mean square error, Hausdorff distance, etc. The differences can include differences in image data. For example, the image data can include grayscale difference, RGB channel difference, or the like. The embodiments of the present disclosure do not limit the manner of obtaining the above differences.

In an embodiment, referring to Formula (1), substituting the first region of interest into

$$R O {I}_{ref}^{i}$$

and the second region of interest into

$${ROI}_{mov}^{i} ,$$

the computer device can determine the first difference Dist according to Formula (2).

$${D}_{1 st} = {\sum }_{i = 1}^{k} {β}_{i} {‖ R O {I}_{ref}^{i} - D ( R O {I}_{mov}^{i} ) ‖}_{2}^{2} . \qquad ( 2 )$$

Similarly, with substituting the first current registration information into D and the first reference registration information into D_{ref}, the computer device can determine the second difference D_{2nd} according to Formula (3).

$${D}_{2 nd} = γ {‖ D - {D}_{ref} ‖}_{2}^{2} . \qquad ( 3 )$$

In an embodiment, the computer device can iterate the first current registration information based on the sum of the first difference and the second difference, and use the iterated first current registration information as new first reference registration information until the first current registration information that meets the first iteration stop condition is taken as the first target registration information.

Taking Formula (1) as an example, let α=0. Based on Formula (1), the computer device iterates the first current registration information D, and uses the iterated first current registration information D as new first reference registration information D_{ref} until the first current registration information D that meets the first iteration stop condition is taken as the first target registration information.

In an embodiment, the first iteration stop condition can be that the number of iterations reaches a preset number, the optimization objective F is less than a preset threshold, or the difference between the optimization objectives F of two adjacent times is less than a preset difference. The embodiments of the present disclosure do not limit the first iteration stop condition.

In the above embodiments, the first difference is determined based on the difference between the first region of interest and the second region of interest, and the second difference is determined based on the difference between the first current registration information and the first reference registration information. Then, the first current registration information is iterated based on the first difference and the second difference, and the iterated first current registration information is used as new first reference registration information, until the first current registration information that meets the first iteration stop condition is taken as the first target registration information. In this way, relatively accurate first target registration information can be determined in the iteration. In addition, during this process, there is no need to pay attention to the full-image information, and more focus is placed on the registration of the regions of interest, which accelerates the determination of the first target registration information.

In an embodiment, as shown in FIG. 8, Step S703 includes Step S801 to Step S802.

In Step S801, a third difference is determined based on the difference between the first floating image and the first reference image.

In an embodiment, referring to Formula (1), substituting the first reference image into I_{ref} and the first floating image into Iₘₒᵥ, the computer device can determine the third difference D_{3rd} according to Formula (4).

$${D}_{3 rd} = α {‖ {I}_{ref} - D ( {I}_{mov} ) ‖}_{2}^{2} . \qquad ( 4 )$$

In some embodiments, the third difference can also be determined based on the difference between the first floating image and the first reference image.

In Step S802, the first current registration information is iterated based on the first difference, the second difference, and the third difference, and the iterated first current registration information is used as new first reference registration information until the first current registration information that meets the first iteration stop condition is taken as the first target registration information.

In this embodiment, the computer device can iterate the first current registration information based on the sum of the first difference, the second difference, and the third difference, and use the iterated first current registration information as new first reference registration information until the first current registration information that meets the first iteration stop condition is taken as the first target registration information.

In an embodiment, referring to Formula (1), the computer device iterates the first current registration information D based on Formula (1), and uses the iterated first current registration information D as new first reference registration information D_{ref}, until the first current registration information D that meets the first iteration stop condition is taken as the first target registration information.

In the above embodiments, the third difference can also be determined based on the difference between the first floating image and the first reference image. Then, the first current registration information is iterated based on the first difference, the second difference, and the third difference, and the iterated first current registration information is used as new first reference registration information, until the first current registration information that meets the first iteration stop condition is taken as the first target registration information. In this way, more accurate first target registration information can be determined.

The following describes the process of determining the second target deformation information.

In an embodiment, the first target registration information includes region-of-interest registration information related to the deformation of the region of interest. Step S202 can be implemented by taking the first target registration information as initial registration information, and the initial registration information is optimized based on the first floating image and the first reference image to obtain the second target registration information.

In this embodiment, the first target registration information includes the region-of-interest registration information related to the deformation of regions of interest. In other words, the region-of-interest registration information can be used for registering at least one region of interest in the first floating image and the corresponding at least one region of interest in the first reference image.

As such, the computer device can take the first target registration information as the initial registration information, and optimize the initial registration information based on the first floating image and the first reference image to obtain the second target registration information.

In an embodiment, optimizing the initial registration information can be a process of iterating or adjusting the initial registration information. The computer device can register the first floating image and the first reference image based on the optimization objective F, and iterate the initial registration information to obtain the second target registration information. Referring to Formula (1), let βᵢ=0. The computer device substitutes the first reference image into I_{ref}, substitutes the first floating image into Iₘₒᵥ, substitutes the first target registration information into D_{ref}, iterates the current registration information D based on Formula (1), and takes D as the second target registration information when the stop condition is met. The stop condition can be that the number of iterations reaches a preset number, or the optimization objective F is less than a preset threshold. The embodiments of the present disclosure do not limit the stop condition.

In some embodiments, the computer device can take the first target registration information as the initial registration information, and optimize the initial registration information based on the second floating image and the second reference image to obtain the second target registration information.

In some embodiments, the computer device can take the first target registration information as the initial registration information, and optimize the initial registration information based on the second floating image, the second reference image, the third region of interest, and the fourth region of interest to obtain the second target registration information.

In the above embodiments, the first target registration information can be used as the initial registration information, and the initial registration information can be optimized based on the first floating image and the first reference image to obtain the second target registration information. As such, by optimizing on the basis of the initial registration information, not only can the effect of the registration of the regions of interest be improved, but also the effect of the registration of the parts other than the regions of interest can be improved, thereby aligning the images.

In an embodiment, as shown in FIG. 9, Step S202 includes Step S901 and Step S902.

In Step S901, second preprocessing is performed on the first floating image and the first reference image to obtain a second floating image and a second reference image, separately.

In this embodiment, the second preprocessing includes, but is not limited to, processes such as window parameter adjustment and/or pixel value normalization. As such, after performing the second preprocessing on the first floating image, the second floating image can be obtained. After performing the second preprocessing on the first reference image, the second reference image can be obtained.

The first preprocessing and the second preprocessing may be the same or different. When the first preprocessing and the second preprocessing are the same, the second floating image is the first preprocessed first floating image, that is, the new first floating image, and the second reference image is the first preprocessed first reference image, that is, the new first reference image.

In Step S902, the second target registration information is determined based on the first target registration information, the second floating image, and the second reference image.

The process of determining the second target registration information in Step S902 is similar to that in Step S202, and will not be repeated here.

In the above embodiments, the second preprocessing can be performed on the first floating image and the first reference image to obtain the second floating image and the second reference image, respectively. Therefore, the first floating image and the first reference image can be correspondingly preprocessed according to requirements. First, the second target registration information can be more efficiently determined based on the first target registration information, the second floating image, and the second reference image. Second, the first preprocessing and the second preprocessing can be different, that is, the images used in determining the first target registration information and the images used in determining the second target registration information can be obtained based on different processings. As such, in the process of determining the first target registration information and the second target registration information, different preprocessing can be performed according to different registration requirements, which improves the accuracy of determining the first target registration information and the second target registration information.

In an embodiment, Step S902 can include that the second target registration information is determined based on the first target registration information, the second floating image, the second reference image, a third region of interest in the second floating image, and a fourth region of interest in the second reference image. The fourth region of interest corresponds to the third region of interest.

In this embodiment, the computer device can also determine the third region of interest in the second floating image and the fourth region of interest in the second reference image. The principle of determining the third region of interest and the fourth region of interest is the same as that of determining the first region of interest and the second region of interest, which can refer to Step S201 and will not be repeated here.

The third region of interest and the first region of interest may be the same or different. The embodiments of the present disclosure do not limit whether the third region of interest and the first region of interest are the same. The fourth region of interest and the second region of interest may be the same or different. The embodiments of the present disclosure do not limit whether the fourth region of interest and the second region of interest are the same.

The computer device can use a second preset deformation algorithm to register the third region of interest and the fourth region of interest, and register the second floating image and the second reference image on the basis of the first target registration information to obtain the second target registration information.

In an embodiment, referring to Formula (1), the computer device substitutes the second reference image into I_{ref}, substitutes the second floating image into Iₘₒᵥ, substitutes the third region of interest into ROIₘₒᵥⁱ, substitutes the fourth region of interest into ROI_{ref}ⁱ, substitutes the first target registration information into D_{ref}, iterates the current registration information D based on Formula (1), and takes the current registration information D as the second target registration information when the stop condition is met.

In the above embodiment, the fourth region of interest corresponds to the third region of interest. Since the second target registration information can be determined based on the first target registration information, the second floating image, the second reference image, the third region of interest in the second floating image, and the fourth region of interest in the second reference image, the region-of-interest information and the full-image information are combined. On the basis of the first target registration information, the second target registration information can be determined more efficiently and accurately.

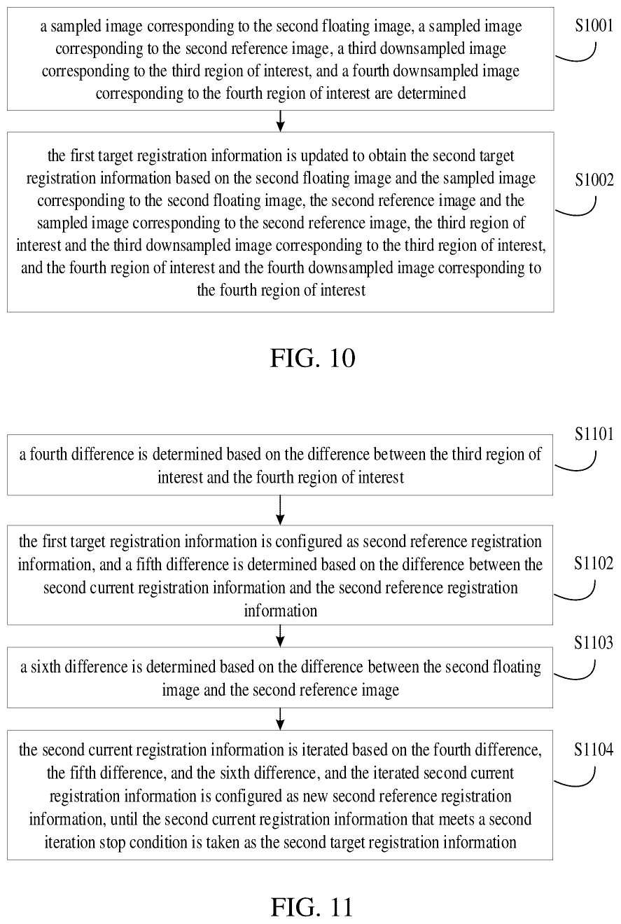

In an embodiment, as shown in FIG. 10, the above step of determining the second target registration information based on the first target registration information, the second floating image, the second reference image, the third region of interest in the second floating image, and the fourth region of interest in the second reference image includes Step S1001 and Step S1002.

In Step S1001, a sampled image corresponding to the second floating image, a sampled image corresponding to the second reference image, a third downsampled image corresponding to the third region of interest, and a fourth downsampled image corresponding to the fourth region of interest are determined.

In this embodiment, the computer device can perform downsampling on the second floating image to obtain the sampled image corresponding to the second floating image, perform downsampling on the second reference image to obtain the sampled image corresponding to the second reference image, perform downsampling on the third region of interest to obtain the third downsampled image, and perform downsampling on the fourth region of interest to obtain the fourth downsampled image.

In Step S1002, the first target registration information is updated to obtain the second target registration information based on the second floating image and the sampled image corresponding to the second floating image, the second reference image and the sampled image corresponding to the second reference image, the third region of interest and the third downsampled image corresponding to the third region of interest, and the fourth region of interest and the fourth downsampled image corresponding to the fourth region of interest.

In an embodiment, the computer device can use a second preset deformation algorithm to register the third downsampled image and the fourth downsampled image, and register the sampled image corresponding to the second floating image and the sampled image corresponding to the second reference image to obtain the initial registration information. Then, the computer device uses the second preset deformation algorithm to register the third region of interest and the fourth region of interest, and registers the second floating image and the second reference image on the basis of the initial registration information to obtain the second target registration information.

In an embodiment, the computer device determines new seventh registration information based on the seventh registration information, the sampled image of the current resolution, the third downsampled image of the current resolution, and the fourth downsampled image of the current resolution. For the historical resolution of the current resolution, the step of determining new seventh registration information based on the seventh registration information, the sampled image of the current resolution, the third downsampled image of the current resolution, and the fourth downsampled image of the current resolution is returned by using the new seventh registration information, until eighth registration information is determined based on the new seventh registration information, the sampled image of the first resolution, the third downsampled image of the first resolution, and the fourth downsampled image of the first resolution. Then, the second target registration information is determined based on the eighth registration information, the second floating image, the second reference image, the third region of interest, and the fourth region of interest. The first one of seventh registration information can be an initial value, such as 0.

In an embodiment, taking the third region of interest 1 and two downsampling operations as an example, after the first downsampling of the second floating image, the second reference image, the third region of interest 1, and the fourth region of interest 1, a sampled image a of the second floating image, a sampled image a of the second reference image, a third downsampled image 1-A, and a fourth downsampled image 1-A are obtained, respectively.

In an embodiment, after the second downsampling of the sampled image a of the second floating image, the sampled image a of the second reference image, the third downsampled image 1-A, and the fourth downsampled image 1-A, a sampled image b of the second floating image, a sampled image b of the second reference image, a third downsampled image 1-B, and a fourth downsampled image 1-B are obtained, respectively.

In an embodiment, the current resolution is the last. Referring to Formula (1), the computer device substitutes the sampled image b of the second reference image into I_{ref}, substitutes the sampled image b of the second floating image into Iₘₒᵥ, substitutes the third downsampled image 1-B into

$$R O {I}_{mov}^{i} ,$$

substitutes the fourth downsampled image 1-B into

$$R O {I}_{ref}^{i} ,$$

substitutes the seventh registration information 1 into D_{ref}, iterates the current registration information D based on Formula (1), and takes the current registration information D as the new seventh registration information 2 when the stop condition is met.

In an embodiment, the computer device substitutes the sampled image a of the second reference image into I_{ref}, substitutes the sampled image a of the second floating image into Iₘₒᵥ, substitutes the third downsampled image 1-A into

$$R O {I}_{mov}^{i} ,$$

substitutes the fourth downsampled image 1-A into

$$R O {I}_{ref}^{i} ,$$

substitutes the seventh registration information 2 into D_{ref}, iterates the current registration information D based on Formula (1), and takes the current registration information D as the eighth registration information when the stop condition is met.

In an embodiment, the computer device substitutes the second floating image into Iₘₒᵥ, substitutes the second reference image into I_{ref}, substitutes the third region of interest 1 into

$$R O {I}_{mov}^{i} ,$$

substitutes the fourth region of interest 1 into

$$R O {I}_{ref}^{i} ,$$

substitutes the eighth registration information into D_{ref}, iterates the current registration information D based on Formula (1), and takes the current registration information D as the second target registration information when the stop condition is met.

In the above embodiments, the sampled image corresponding to the second floating image, the sampled image corresponding to the second reference image, the third downsampled image corresponding to the third region of interest, and the fourth downsampled image corresponding to the fourth region of interest can be determined. The first target registration information is updated to obtain the second target registration information based on the second floating image and the sampled image corresponding to the second floating image, the second reference image and the sampled image corresponding to the second reference image, the third region of interest and the third downsampled image corresponding to the third region of interest, and the fourth region of interest and the fourth downsampled image corresponding to the fourth region of interest. Therefore, the accuracy of the second target registration information can be further improved through downsampling.

In an embodiment, as shown in FIG. 11, the above step of determining the second target registration information based on the first target registration information, the second floating image, the second reference image, the third region of interest in the second floating image, and the fourth region of interest in the second reference image includes Step S1101 to Step S1104.

In Step S1101, a fourth difference is determined based on the difference between the third region of interest and the fourth region of interest.

In an embodiment, taking Formula (1) as an example, substituting the third region of interest into

$$R O {I}_{mov}^{i}$$

and the fourth region of ineterest into

$$R O {I}_{ref}^{i} ,$$

the computer device can determine the fourth difference D₄ₜₕ according to Formula (5).

$${D}_{4 th} = {\sum }_{i = 1}^{k} {β}_{i} {‖ R O {I}_{ref}^{i} - D ( R O {I}_{mov}^{i} ) ‖}_{2}^{2} . \qquad ( 5 )$$

In Step S1102, the first target registration information is used as second reference registration information, and a fifth difference is determined based on the difference between the second current registration information and the second reference registration information.

Similarly, substituting the second current registration information into D and the second reference registration information into D_{ref}, the computer device can determine the fifth difference based on the second difference.

In Step S1103, a sixth difference is determined based on the difference between the second floating image and the second reference image.

In an embodiment, the computer device can substitute the second reference image into I_{ref} and the second floating image into Iₘₒᵥ, so that the computer device can determine the sixth difference based on the third difference.

In Step S1104, the second current registration information is iterated based on the fourth difference, the fifth difference, and the sixth difference, and the iterated second current registration information is used as new second reference registration information, until the second current registration information that meets a second iteration stop condition is taken as the second target registration information.

In an embodiment, the computer device can iterate the second current registration information based on the sum of the fourth difference, the fifth difference, and the sixth difference, and use the iterated second current registration information as new second reference registration information, until the second current registration information that meets the second iteration stop condition is taken as the second target registration information.

In an embodiment, the second iteration stop condition can also be that the number of iterations reaches a preset number, the optimization objective F is less than a preset threshold, or the difference between the optimization objectives F of two adjacent times is less than a preset difference. The first iteration stop condition and the second iteration stop condition may be the same or different.

Referring to Formula (1), the computer device iterates the second current registration information D based on Formula (1), and uses the iterated second current registration information D as new second reference registration information D_{ref}, until the second current registration information D that meets the second iteration stop condition is taken as the second target registration information.

In the above embodiments, the fourth difference can be determined based on the difference between the third region of interest and the fourth region of interest, the fifth difference can be determined based on the difference between the second current registration information and the second reference registration information, and the sixth difference can be determined based on the difference between the second floating image and the second reference image. Then, the first target registration information is used as the second reference registration information, the second current registration information is iterated based on the fourth difference, the fifth difference, and the sixth difference, and the iterated current registration information is used as new second reference registration information, until the second current registration information that meets the second iteration stop condition is taken as the second target registration information. As such, the relatively accurate second target registration information can be determined through continuous iteration.

In conclusion, the technical solution of the present invention addresses the technical problem of simultaneous deformation of the region of interest and the image through two or more processes of deformable registration. The first deformation achieves better registration of the regions of interest through various preprocessing steps. The second deformation takes the registration information obtained from the first deformation as the initial registration information, and adds the part of the regions of interest in the initial registration information as reference registration information to the optimization function. During the optimization, while ensuring that the registration information of the regions of interest remains basically unchanged, the other parts of the image, except the regions of interest, are deformed.

At present, in some application scenarios, with the development of radiotherapy technology and the integration of different modal imaging technologies with radiotherapy, new radiotherapy technologies such as online adaptive radiotherapy and dose-guided radiotherapy have attracted increasing attention.

Online adaptive radiotherapy can address the technical problems of target volume movement between different treatment fractions, lesion regression and changes, and continuous changes of the target volume affected by surrounding organs during treatment fractions, realizing precise treatment for patients having a tumor, reducing the toxicity and side effects after radiotherapy, and improving the quality of life of patients. Dose-guided radiotherapy needs to calculate the dose in time under the current posture of patients to apply radiotherapy under dose guidance. Both online adaptive radiotherapy and dose-guided radiotherapy have high requirements for the time of the entire process. Only when the time is short can uncertainties of dose caused by factors such as organ movement be reduced. Therefore, shortening the calculation time of the dose as much as possible is a key research goal. Based on this, the present disclosure further provides a dosage determination method, which will be described below.

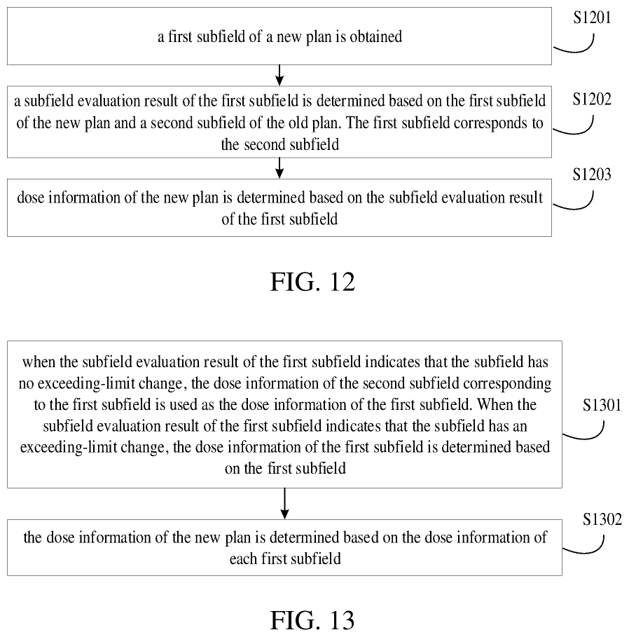

In an embodiment, as shown in FIG. 12, a dosage determination method is provided. Taking the dosage determination method applied to the computer device in FIG. 1 as an example, the dosage determination method includes the following Step S1201 to Step S1203.

In Step S1201, a first subfield of a new plan is obtained.

In an embodiment, the computer device can receive the new plan sent by a Treatment Planning System (TPS) and determine the first subfield of the new plan. After obtaining the first subfield of the new plan, the computer device also determines the size and shape of the first subfield. For example, the new plan can refer to a radiotherapy plan corresponding to a first radiotherapy time period, and an old plan can refer to a radiotherapy plan corresponding to a second radiotherapy time period. At least a part of the first radiotherapy time period is later than the second radiotherapy time period.

In an embodiment, when radiotherapy includes multiple treatment fractions, the new plan can be a radiotherapy plan formulated for the current treatment fraction. The old plan can be an initially formulated radiotherapy plan or a radiotherapy plan formulated for a previous historical treatment fraction.

In some embodiments, the radiotherapy plan can be obtained by registering the floating image and the reference image based on the above image registration method, and the registered floating image and reference image. In other words, the new plan or the old plan can be obtained by registering the floating image and the reference image based on the above image registration method, and the registered floating image and reference image.

In an embodiment, a first medical image of the subject at a first time point can be used as the reference image. A second medical image of the subject at a second time point can be used as the floating image. The reference image and the floating image are registered based on the above image registration method. The radiotherapy plan is obtained based on the reference image and the floating image. The first time point is later than the second time point.

In Step S1202, a subfield evaluation result of the first subfield is determined based on the first subfield of the new plan and a second subfield of the old plan. The first subfield corresponds to the second subfield.

In an embodiment, the computer device can also determine the second subfield of the old plan.

In an embodiment, the computer device obtains the second subfield of the old plan from a storage space. The storage space can be a storage space on the computer device or a storage space on another storage device, such as a server. The embodiments of the present disclosure do not limit implementations of the storage space.

In an embodiment, the new plan includes at least one first subfield. The old plan includes at least one second subfield. The first subfield corresponds to the second subfield. A subfield can be a small radiation field with a certain shape that allows radiation to pass through. In some embodiments, an overlap of the shapes of multiple subfields (which may be called a radiation field) can match the shape of the target volume.

In an embodiment, the shape, size, and the like of the subfield can be adjusted through the grating leaves of a multi-leaf collimator. The first subfield and the second subfield formed by the same one or more grating leaves correspond to each other. However, the manner of forming or adjusting the subfield is not limited thereto, and other types of collimators can also be used to form or adjust the subfield. For example, a collimator with fixed openings of different shapes and sizes can be used. By switching the positions of the fixed openings relative to a radiation source, the shape and size of the radiation beam passing through the collimator can be formed or adjusted, so as to realize the formation or adjustment of the subfield.

In an embodiment, the grating leaves include Leaf 1, Leaf 2, and Leaf 3. Then the new plan may include a first subfield 1 corresponding to Leaf 1, a first subfield 2 corresponding to Leaf 2, and a first subfield 3 corresponding to Leaf 3. The old plan may include a second subfield 1 corresponding to Leaf 1, a second subfield 2 corresponding to Leaf 2, and a second subfield 3 corresponding to Leaf 3. The first subfield 1 corresponds to the second subfield 1, the first subfield 2 corresponds to the second subfield 2, and the first subfield 3 corresponds to the second subfield 3.

In an embodiment, the computer device can determine the subfield evaluation result of the first subfield based on the first subfield of the new plan and the second subfield of the old plan. The computer device can determine the subfield evaluation result of the first subfield 1 based on the first subfield 1 and the second subfield 1, determine the subfield evaluation result of the first subfield 2 based on the first subfield 2 and the second subfield 2, and determine the subfield evaluation result of the first subfield 3 based on the first subfield 3 and the second subfield 3.

In an embodiment, the subfield evaluation result of the first subfield can characterize whether the first subfield and the corresponding second subfield have an exceeding-limit change.

In an embodiment, the subfield evaluation result of the first subfield can further include a degree of change between the first subfield and the corresponding second subfield. For example, the degree of change can be a value between 0 and 1. The smaller the value is, the smaller the degree of change.

In an embodiment, the computer device can determine whether the position of a grating leaf has an exceeding-limit change through a sensor, so as to determine the subfield evaluation result of the first subfield. The computer device can also determine the subfield evaluation result of the first subfield by comparing the shapes and sizes of the first subfield and the corresponding second subfield, which is not limited in the embodiments.

In Step S1203, dose information of the new plan is determined based on the subfield evaluation result of the first subfield.

In an embodiment, the computer device can determine the dose information of the new plan based on the subfield evaluation result of the first subfield. For example, when the degree of change between the first subfield and the corresponding second subfield is less than a first threshold, the computer device uses the dose information of the second subfield as the dose information of the corresponding first subfield. When the degree of change between the first subfield and the corresponding second subfield is not less than the first threshold but less than a second threshold, the computer device adjusts the dose information of the second subfield to obtain the dose information of the corresponding first subfield. When the degree of change between the first subfield and the corresponding second subfield is not less than the second threshold, the computer device recalculates the dose information of the first subfield based on the first subfield.

At present, in the related art, as long as the plan changes or has an exceeding-limit change, it is necessary to recalculate the dose information of all subfields in the new plan. However, recalculating the dose information of all subfields in the new plan takes a long time. In the above dosage determination method, the first subfield corresponds to the second subfield. Since the first subfield of the new plan can be obtained, and the subfield evaluation result of the first subfield can be determined based on the first subfield of the new plan and the second subfield of the old plan, the dose information of the new plan can be efficiently determined based on the subfield evaluation result of the first subfield without recalculating the dose information of all first subfields. Therefore, the time for dose determination can be reduced, and the efficiency of dose determination can be improved. Especially for adaptive radiotherapy, it is necessary to readjust or formulate a radiotherapy plan between every two treatment fractions. Through the dosage determination method of the present disclosure, the time consumed for the calculation of dose information can be significantly reduced.

In an embodiment, as shown in FIG. 13, Step S1203 includes Step S1301 to Step S1302.

In Step S1301, when the subfield evaluation result of the first subfield indicates that the subfield has no exceeding-limit change, the dose information of the second subfield corresponding to the first subfield is used as the dose information of the first subfield. When the subfield evaluation result of the first subfield indicates that the subfield has an exceeding-limit change, the dose information of the first subfield is determined based on the first subfield.

In an embodiment, the computer device can obtain the dose information of the second subfield.

In an embodiment, the computer device can obtain the dose information of the second subfield from the storage space. The dose information is used to indicate a dose distribution of the corresponding subfield. Dose information includes, but is not limited to, a dose field, a flux map, and a spatial dose model.

The term “exceeding-limit change” used herein can refer to a change exceeding a predetermined limit. For example, when the allowable limit is 0, the exceeding-limit change can include no change at all. When the allowable limit is a predetermined degree (such as +1% or a specific value of difference), the exceeding-limit change can include a degree of change greater than the predetermined degree (such as a degree of change greater than +1%).

In an embodiment, taking the first subfield 1 as an example, when the subfield evaluation result of the first subfield 1 indicates that the subfield has no exceeding-limit change, it means that the first subfield 1 has not changed or has basically not changed relative to the second subfield 1 (for example, a change range or difference of the first subfield 1 relative to the second subfield 1 is less than a preset change threshold or a preset difference threshold, such as the relative difference between the first subfield 1 and the second subfield 1 is within +10%, +3%, +1%, etc.). Thus, the dose information of the second subfield 1 is still applicable to the first subfield 1. The computer device further uses the dose information corresponding to the second subfield 1 as the dose information of the first subfield 1.

In an embodiment, taking the first subfield 1 as an example, when the subfield evaluation result of the first subfield 1 indicates an exceeding-limit change, it means that the first subfield 1 has changed relative to the second subfield 1 or has a change exceeding the preset change threshold. Therefore, the dose information of the second subfield 1 is not applicable to the first subfield 1. Thus, the computer device recalculates the dose information of the first subfield, that is, determines the dose information of the first subfield 1 based on the first subfield 1. This embodiment does not limit the process of determining the dose information of the first subfield based on the first subfield.

In Step S1302, the dose information of the new plan is determined based on the dose information of each first subfield.

In an embodiment, after determining the dose information of each first subfield, the computer device can determine the dose information of the new plan. For example, the computer device can superimpose the dose information of each first subfield to obtain the dose information of the new plan.

In the above embodiments, when the subfield evaluation result of the first subfield indicates no exceeding-limit change, the dose information of the second subfield corresponding to the first subfield is used as the dose information of the first subfield. When the subfield evaluation result of the first subfield indicates an exceeding-limit change, the dose information of the first subfield is determined based on the first subfield. As such, for the first subfields that have not changed or have a degree of change less than the threshold, there is no need to calculate the corresponding dose information, and the dose information of the corresponding second subfield can be directly used. Therefore, the dose information of the new plan can be efficiently determined based on the dose information of each first subfield.

In an embodiment, Step S1302 can be implemented as when the difference between the first subfield and the second subfield corresponding to the first subfield is greater than a preset difference, it is determined that the subfield evaluation result of the first subfield is an exceeding-limit change of the subfield.

In this embodiment, the above difference can be a shape difference or an area difference between the first subfield and the second subfield corresponding to the first subfield. The embodiments of the present disclosure do not limit these shape differences or area differences.

In an embodiment, when the difference between the leaf position information corresponding to the first subfield and leaf position information corresponding to the second subfield is greater than a preset difference, the computer device determines that the subfield evaluation result of the first subfield is an exceeding-limit change of the subfield. On the contrary, when the difference between leaf position information corresponding to the first subfield and the leaf position information corresponding to the second subfield is not greater than the preset difference, the computer device determines that the subfield evaluation result of the first subfield is not an exceeding-limit change of the subfield. The preset difference can be set according to requirements.

In an embodiment, the leaf position information refers to a position where a grating leaf is located. For example, the computer device can obtain the leaf position information corresponding to the first subfield and the leaf position information corresponding to the second subfield through a sensor. As such, by comparing whether the difference between the leaf position information corresponding to the first subfield and the leaf position information corresponding to the second subfield is greater than the preset difference, the computer device can efficiently determine whether the subfield evaluation result of the first subfield is an exceeding-limit change.

In the above embodiment, since it can be determined that the subfield evaluation result of the first subfield is an exceeding-limit change of the subfield when the difference between the first subfield and the second subfield corresponding to the first subfield is greater than the preset difference, it can be efficiently determined whether to continue using the dose information of the second subfield through the subfield evaluation result, thereby accelerating the speed of dose determination.

In an embodiment, the above dosage determination method further includes storing the first subfield and the dose information of the first subfield.

In this embodiment, after determining the new plan, the computer device stores the first subfield and the dose information of the first subfield. This embodiment does not limit the manner of storage or the location of storage. As such, after the new plan is modified, Step S1201 to Step S1203 can be continued to obtain the dose information of the modified new plan.

In the above embodiment, since the first subfield and the dose information of the first subfield can be stored, it is beneficial for the next round of modification of the new plan.

In an embodiment, the above dosage determination method further includes obtaining the first subfield in response to a triggered modification instruction.

In this embodiment, a modification instruction is triggered when the old plan is modified, and the computer device can receive the modification instruction and obtain the first subfield in response to the triggered modification instruction. For example, the computer device can obtain the first subfield in response to the triggered modification instruction under the action of online adaptive radiotherapy and dose-guided radiotherapy. As such, the corresponding dose information can be determined in time when the radiotherapy plan has an exceeding-limit change.

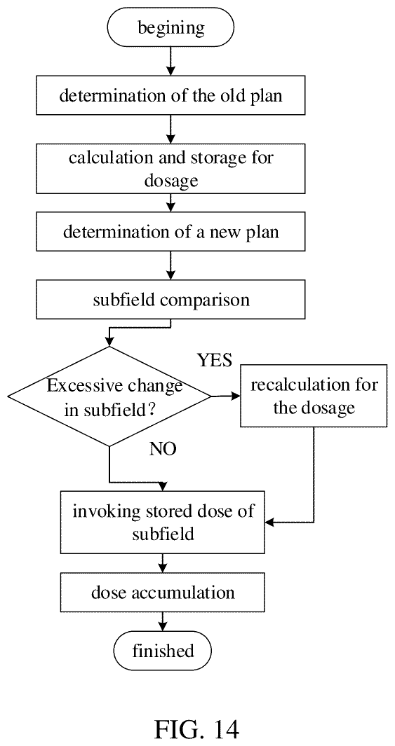

In an embodiment, as shown in FIG. 14, the computer device first determines the old plan, calculates and stores a dosage for the old plan, that is, determines the second subfield of the old plan and the corresponding dose information of the second subfield. After modifying the old plan, the computer device can determine the new plan, that is, determine the first subfield corresponding to the new plan. Further, the computer device performs a comparison of subfields to determine whether a subfield has an exceeding-limit change, that is, determines the subfield evaluation result of the first subfield based on the first subfield of the new plan and the second subfield of the old plan. When the subfield has an exceeding-limit change, the computer device performs dosage recalculation, that is, when the subfield evaluation result of the first subfield indicates an exceeding-limit change of the subfield, the dose information of the first subfield is determined based on the first subfield. When the subfield has no exceeding-limit change, the computer device directly invokes a stored dose of the subfield, that is, when the subfield evaluation result of the first subfield indicates no exceeding-limit change of the subfield, the dose information of the second subfield corresponding to the first subfield is used as the dose information of the first subfield. After dose superimpose, the dose information of the new plan is obtained.

For radiotherapy, both image registration and dose calculation are very important. The image registration method and dosage determination method applicable to adaptive radiotherapy provided in the present disclosure can shorten the time for the adaptive treatment and improve the efficiency of the adaptive treatment.

Although the steps in the flowcharts involved in the above embodiments are sequentially displayed according to the arrows, these steps are not necessarily executed sequentially in the order indicated by the arrows. Unless clearly stated in this document, the execution of these steps is not strictly limited in order, and these steps can be executed in other orders. In addition, at least some of the steps in the flowcharts involved in the above embodiments can include multiple steps or multiple stages. These steps or stages are not necessarily executed at the same time, but can be executed at different times. The execution order of these steps or stages is not necessarily sequential, but can be executed in turn or alternately executed with at least a part of the steps or stages in other steps or other stages.

Based on the same inventive concept, an embodiment of the present disclosure further provides an image registration apparatus for implementing the above image registration method. The technical solution for addressing the technical problem provided by this apparatus is similar to the technical solution recited in the above image registration method. Therefore, the specific definition in one or more embodiments of the image registration apparatus provided below can refer to the definition of the image registration method above, and will not be repeated here.

In an embodiment, as shown in FIG. 15, an image registration apparatus 1500 is provided. The image registration apparatus 1500 includes a first determination module 1501 and a second determination module 1502.

The first determination module 1501 is configured to determine first target registration information based on the first region of interest in the first floating image and the second region of interest in the first reference image. The second region of interest corresponds to the first region of interest. The first target registration information is used for registering the first region of interest with the second region of interest.

The second determination module 1502 is configured to determine the second target registration information based on the first target registration information, the first floating image, and the first reference image. The second target registration information is used for registering the first floating image with the first reference image.

In the above image registration apparatus, the first target registration information is determined based on the first region of interest in the first floating image and the second region of interest in the first reference image. The second region of interest corresponds to the first region of interest. Since the first target registration information is used for registering the first region of interest with the second region of interest, the first target registration information is first obtained by focusing only on the registration of the regions of interest. Then, the second target registration information is determined based on the first target registration information, the first floating image, and the first reference image. As such, on the basis of the first target registration information, registration can be executed based on the entire first floating image and the entire first reference image, so that not only can the regions of interest of the two images be well registered, but the parts other than the regions of interest in the two images can also be well registered. Thus, the first floating image and the first reference image can be accurately registered through the second target registration information.

Based on the same inventive concept, a dosage determination apparatus for implementing the above dosage determination method is further provided in an embodiment of the present disclosure. The technical solution for addressing the technical problem provided by this dosage determination apparatus is similar to the technical solution recited in the above dosage determination method. Therefore, the specific definition in one or more embodiments of the dosage determination apparatus provided below can refer to the definition of the dosage determination method above, and will not be repeated here.

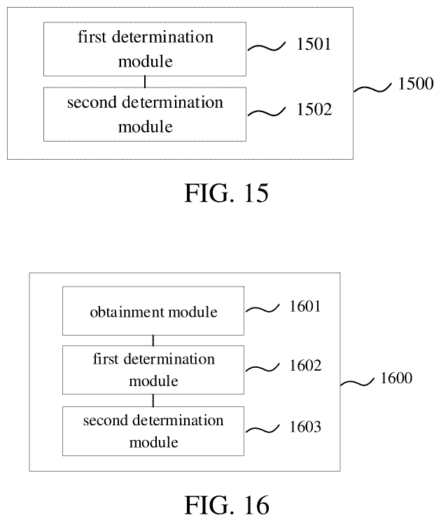

In an embodiment, as shown in FIG. 16, a dosage determination apparatus 1600 is provided. The dosage determination apparatus 1600 includes an obtainment module 1601, a first determination module 1602, and a second determination module 1603.

The obtainment module 1601 is configured to obtain the first subfield of the new plan.

The first determination module 1602 is configured to determine the subfield evaluation result of the first subfield based on the first subfield of the new plan and the second subfield of an old plan. The first subfield corresponds to the second subfield.

The second determination module 1603 is configured to determine the dose information of the new plan based on the subfield evaluation result of the first subfield.

Each module in the above image registration apparatus or dosage determination apparatus can be fully or partially implemented in software, hardware, or a combination thereof. Each of the above modules can be embedded in or independent of a processor of the computer device in hardware, or stored in a memory of the computer device in software, so that the processor can invoke and execute the operations corresponding to each of the above modules.

In an embodiment, a computer device is provided. The computer device includes a memory and a processor. The memory stores a computer program. The processor, when executing the computer program, implements the methods in the above embodiments.

In an embodiment, a non-transitory computer-readable storage medium is provided, on which a computer program is stored. The computer program, when executed by a processor, implements the methods provided in the above embodiments.

In an embodiment, a computer program product is provided. The computer program product includes a computer program. The computer program, when executed by a processor, implements the methods provided in the above embodiments.

Those of ordinary skill in the art can understand that all or part of the processes in the methods provided in the above embodiments can be completed by instructing relevant hardware through a computer program. The computer program can be stored in a non-transitory computer-readable storage medium. The computer program, when executed, implements the methods provided in the above embodiments. Any reference to the memory, a database, or other media involved in the embodiments provided in the present disclosure can include at least one of a non-volatile memory or a volatile memory. The non-volatile memory can include a Read-Only Memory (ROM), a magnetic tape, a floppy disk, a flash memory, an optical memory, a high-density embedded non-volatile memory, a Resistive Random Access Memory (ReRAM), a Magnetoresistive Random Access Memory (MRAM), a Ferroelectric Random Access Memory (FRAM), a Phase Change Memory (PCM), a graphene memory, etc. The volatile memory can include a Random Access Memory (RAM), an external cache memory, etc. For explanation and not limitation, the RAM may be in various forms, such as a Static Random Access Memory (SRAM) or a Dynamic Random Access Memory (DRAM). The database involved in the embodiments provided in the present disclosure may include at least one of a relational database or a non-relational database. The non-relational database can include such as a blockchain-based distributed database. The processor provided in the embodiments of the present disclosure can be a general-purpose processor, a central processing unit, a graphics processor, a digital signal processor, a programmable logic device, a quantum computing-based data processing logic device, an Artificial Intelligence (AI) processor, etc.

The various technical features of the above embodiments can be arbitrarily combined. For the sake of a concise description, not all possible combinations of the various technical features in the above embodiments are described. However, as long as there is no contradiction in the combination of these technical features, they should be regarded as falling within the scope of the present disclosure.

The above embodiments only express several implementations of the present disclosure, and their descriptions are relatively specific and detailed, but they should not be construed as limiting the scope of the present disclosure. For those of ordinary skill in the art, without departing from the inventive concept of the present disclosure, several modifications and improvements can be made, and these modifications and improvements all fall within the scope of the present disclosure.

What is claimed is:

1. An image registration method, comprising:
determining first target registration information based on a first region of interest in a first floating image and a second region of interest in a first reference image, wherein the second region of interest corresponds to the first region of interest, and the first target registration information is configured for registering the first region of interest with the second region of interest; and
determining second target registration information based on the first target registration information, the first floating image, and the first reference image, wherein the second target registration information is configured for registering the first reference image with the first floating image.

2. The image registration method according to claim 1, wherein determining the first target registration information based on the first region of interest in the first floating image and the second region of interest in the first reference image comprises:
identifying the first region of interest in the first floating image, identifying the second region of interest in the first reference image, registering the first region of interest and the second region of interest, and determining the first target registration information.

3. The image registration method according to claim 2, wherein identifying the first region of interest in the first floating image, identifying the second region of interest in the first reference image, registering the first region of interest and the second region of interest, and determining the first target registration information comprises:
determining a first downsampled image corresponding to the first region of interest and a second downsampled image corresponding to the second region of interest;
updating first registration information based on the first downsampled image and the second downsampled image;
iteratively updating the first registration information based on historical information to determine second registration information; and
determining the first target registration information based on the second registration information, the first region of interest, and the second region of interest.

4. The image registration method according to claim 1, wherein the first region of interest comprises a plurality of first regions of interest, the second region of interest comprises a plurality of second regions of interest, and determining the first target registration information based on the first region of interest in the first floating image and the second region of interest in the first reference image comprises:
determining third registration information based on a first area and a second area corresponding to the first area, wherein the first area comprises at least one of the plurality of first regions of interest, and the second area comprises at least one of the plurality of second regions of interest; and
determining the first target registration information based on the third registration information, a third area, and a fourth area corresponding to the third area, wherein the third area comprises at least one of the plurality of first regions of interest other than the first area, and the fourth area comprises at least one of the plurality of second regions of interest other than the second area.

5. The image registration method according to claim 4, further comprising:
formulating a first optimization function based on the third registration information, the third area, and the fourth area, wherein the fourth area corresponds to the third area; and
determining the first target registration information through at least one iteration based on the first optimization function.

6. The image registration method according to claim 5, wherein formulating the first optimization function comprises formulating a regularization term of the first optimization function based on the third registration information.

7. The image registration method according to claim 4, wherein determining the first target registration information based on the third registration information, the third area, and the fourth area corresponding to the third area comprises determining the first target registration information through at least one iteration based on the third registration information, the third area, and the fourth area corresponding to the third area.

8. The image registration method according to claim 7, wherein formulating the first optimization function comprises formulating a regularization term of the first optimization function based on the third registration information.

9. The image registration method according to claim 1, wherein determining the first target registration information based on the first region of interest in the first floating image and the second region of interest in the first reference image comprises determining the first target registration information based on the first region of interest, the second region of interest, the first floating image, and the first reference image.

10. The image registration method according to claim 9, wherein weights of the first floating image and the first reference image are less than weights of the first region of interest and the second region of interest in determining the first target registration information.

11. The image registration method according to claim 1, wherein determining the first target registration information based on the first region of interest in the first floating image and the second region of interest in the first reference image comprises:
determining a first difference between the first region of interest and the second region of interest;
determining a second difference between first current registration information and first reference registration information;
formulating a first optimization function based on the first difference and the second difference; and
determining the first target registration information through at least one iteration based on the first optimization function.

12. The image registration method according to claim 11, wherein formulating the first optimization function based on the first difference and the second difference, and determining the first target registration information through at least one iteration based on the first optimization function comprises:
determining a third difference between the first floating image and the first reference image; and
iterating the first current registration information based on the first difference, the second difference, and the third difference to obtain iterated first current registration information that meets a first iteration stop condition, and the iterated first current registration information is taken as the first target registration information.

13. The image registration method according to claim 1, wherein the first target registration information comprises region-of-interest registration information related to deformation of regions of interest, and determining the second target registration information based on the first target registration information, the first floating image, and the first reference image comprises:
obtaining the second registration information by optimizing initial registration information based on the first floating image and the first reference image with the first target registration information serving as the initial registration information.

14. The image registration method according to claim 1, wherein before determining first target registration information based on a first region of interest in a first floating image and a second region of interest in a first reference image, the method further comprises:
separately performing a first preprocessing on the first floating image and the first reference image to obtain a preprocessed first floating image and a preprocessed first reference image, wherein the first preprocessing comprises window parameter adjustment and/or pixel value normalization.

15. The image registration method according to claim 1, wherein determining the second target registration information based on the first target registration information, the first floating image, and the first reference image comprises:
separately performing a second preprocessing on the first floating image and the first reference image to obtain a second floating image and a second reference image, wherein the second preprocessing comprises window parameter adjustment and/or pixel value normalization; and
determining a second target registration information based on the first target registration information, the second floating image, and the second reference image.

16. The image registration method according to claim 15, wherein determining the second target registration information based on the first target registration information, the second floating image, and the second reference image comprises:
determining the second target registration information based on the first target registration information, the second floating image, the second reference image, a third region of interest identified in the second floating image, and a fourth region of interest identified in the second reference image, wherein the fourth region of interest corresponds to the third region of interest.

17. The image registration method according to claim 16, wherein determining the second target registration information based on the first target registration information, the second floating image, the second reference image, the third region of interest identified in the second floating image, and the fourth region of interest identified in the second reference image comprises:
determining a sampled image corresponding to the second floating image, a sampled image corresponding to the second reference image, a third downsampled image corresponding to the third region of interest, and a fourth downsampled image corresponding to the fourth region of interest; and
updating the first target registration information to obtain the second target registration information based on the second floating image and the sampled image corresponding to the second floating image, the second reference image and the sampled image corresponding to the second reference image, the third region of interest and the third downsampled image corresponding to the third region of interest, and the fourth region of interest and the fourth downsampled image corresponding to the fourth region of interest.

18. The image registration method according to claim 16, wherein determining the second target registration information based on the first target registration information, the second floating image, the second reference image, the third region of interest identified in the second floating image, and the fourth region of interest identified in the second reference image comprises:
determining a fourth difference between the third region of interest and the fourth region of interest;
determining a fifth difference between second current registration information and second reference registration information by taking the first target registration information as the second reference registration information;
determining a sixth difference between the second floating image and the second reference image; and
iterating the second current registration information based on the fourth difference, the fifth difference, and the sixth difference to obtain iterated second current registration information that meets a second iteration stop condition, and the iterated second current registration information is taken as the second target registration information.

19. A dosage determination method, comprising:
obtaining a first subfield corresponding to a new plan;
determining a subfield evaluation result of the first subfield according to the first subfield corresponding to the new plan and a second subfield corresponding to an old plan, wherein the first subfield corresponds to the second subfield.

20. A non-transitory computer-readable storage medium, having a computer program stored thereon, wherein the computer program, when executed by a processor, implements the image registration method according to claim 1.
